(19) United States
(12) Patent Application Publication
(10) Pub. No.: US 2011/0240011 A1
(43) Pub. Date: Oct. 6, 2011

(54) Treating apparatus

(75) Inventor: Michael V. Caldwell, Coarsegold, CA (US)

(73) Assignee: Elivn L. Haworth

(21) Appl. No.: 12/798,224

(22) Filed: Mar. 31, 2010

(51) Int. Cl. A61M 11/00

(52) U.S. Cl. 128/200.14

(57) ABSTRACT

A treating apparatus having a source of a flowable substance, an assembly for releasing the flowable substance in a general direction relative to a target, and a propelling assembly operable to transport the flowable substance from the source to the releasing means whereby the flowable substance is propelled in the general direction relative to the target.

### CROSS-REFERENCE TO RELATED APPLICATIONS

Not applicable.

### STATEMENT REGARDING FEDERALLY SPONSORED RESEARCH OR DEVELOPMENT

Not Applicable.

### BACKGROUND OF THE INVENTION

(1) Field of the Invention

The present invention relates to a treating apparatus and, more particularly, to a treating apparatus which is adapted to a wide variety of uses including those relating to a person's health.

(2) Description of the Prior Art

Medical technology has advanced at an ever increasing rate over many decades. These advances cover a broad spectrum of specific technologies. Simultaneously, otherwise unrelated technologies have also advanced at a rapid pace. While the foregoing advances have, in many cases, been not less than miraculous, there are many areas in which this technology has not been applied, or is otherwise lagging in development.

There has been a lack of recognition of the uses to which these technologies could be directed, or of the benefits to be derived therefrom. For example, very little progress has been accomplished where communicable diseases may exist in public environments. This is particularly the case where a great number of people may be placed in close contact, have contact with suspected contaminated areas, substances or the like. Such public environments exist, for example, in public transportation, restaurants, city streets, on commercial aircraft, in theaters, at sports events, or any other large gatherings of people, or by exposure to people who may have a higher possibility of being infected, such as in doctor's offices, hospitals and other health care facilities.

Conventional practice in such areas of exposure is most frequently to take no precautions whatsoever. In a small number of cases, people may wear surgical masks, or similar facial coverings, for the purpose of limiting such exposure. Such measures are largely self-defeating for numerous reasons. Only a few such reasons need be mentioned to illustrate the point. Surgical masks are porous so that contaminants can pass in both directions therethrough. To the extent they capture contaminants, the contaminants are actually retained in the optimum position to cause exposure; that is, covering the nose and mouth of the person wearing the mask. They are not sealed around the edges so they provide no protection whatsoever to the passage of contaminants around the edges of the masks. Further, such masks are frequently reused with all of the attendant contamination associated therewith.

In short, no remotely adequate method, or apparatus, is conventionally available for avoiding the direct spread of communicable diseases by such exposure.

The lack of utilizing technology to full advantage is also illustrated by another example. Monitoring people's health from remote locations has not utilized technology. For example, medication is usually prescribed for a person, based upon the results of the persons last in office medical examination. The results of the examination direct the attending physician to prescribe those medications which appear indicated. There is no provision available to deal with sudden changes in the physical condition of the patient. Without attention, people otherwise under a doctor's care can have life threatening events with no adequate possibility of prompt treatment, or, for that matter, the doctor even being aware of the event. As a consequence, needless injury, or death, is well within the realm of possibility.

Still another example of the lack of the application of technology to the solution of existing problems relates to a persons vital signs. The pulse rate, heart rate and substantially more vital information is not available, as a practical matter, either to the person, doctor, or other health care workers because these vital signs are usually only taken during an in office doctor's appointment, or at the time of some type of medical emergency. Continuous monitoring of such information is largely unknown. As a consequence, serious injury or death may unnecessarily transpire. Regret frequently takes the form of wishing that the event could have been anticipated and medical personnel intervened to attempt to avoid the result.

Therefore, it has long been known that it would be desirable to have a treating apparatus which provides protection against the spread of communicable diseases and the like; which is unobtrusive when worn by a person and yet which operates reliably and with an effectiveness not heretofore achieved in the art; which affords a flexibility of use allowing it to be tailored for the specific needs of the subject; which affords a broad range of benefits in use and yet which possesses a simplicity and adaptability in use; which is fully operational in a variety of embodiments; and which is otherwise entirely successful in achieving its operational objectives.

### BRIEF SUMMARY OF THE INVENTION

Therefore, it is an object of the present invention to provide an improved treating apparatus.

Another object is to provide such a treating apparatus which substantially improves upon the ability to avoid the spread of communicable diseases and the like.

Another object is to provide such a treating apparatus which is adaptable to a broad range of embodiments based upon the objectives desired to be achieved.

Another object is to provide such a treating apparatus which is particularly well suited to the reliable prevention of the spread of communicable diseases.

Another object is to provide such a treating apparatus which is unobtrusive in use.

Another object is to provide such a treating apparatus which is adaptable for both protecting the subject from exposure to communicable diseases as well as to protect the public form exposure to that of the subject.

Another object is to provide such a treating apparatus which is capable of providing the rapid transmission of health data about a person to a designated party such as a doctor, a hospital or other health care facility.

Another object is to provide such a treating apparatus which is of uncomplicated and inexpensive construction permitting wide usage thereof.

Another object is to provide such a treating apparatus which permits the operator to have immediate access to a plurality of medicinal substances substantially instantaneously under the person's control and with his knowledge.

Further objects and advantages are to provide improved elements and arrangements thereof in an apparatus for the purposes described which is dependable, economical, durable and fully effective in accomplishing its intended purposes.

These and other objects and advantages are achieved, in the preferred embodiment of the apparatus of the present invention, in a treating apparatus having a source of a flowable substance; a releasing mechanism for releasing the flowable substance in a general direction relative to a target; and a propelling assembly operable to transport the flowable substance from said source to said releasing mechanism whereby said flowable substance is propelled in a given direction relative to the target.

### BRIEF DESCRIPTION OF THE SEVERAL VIEWS OF THE DRAWINGS

FIG. 1 is a fragmentary front elevation of the first embodiment of the treating apparatus of the present invention shown in a typical operative environment being worn by a person shown in full lines.

FIG. 2 is a somewhat enlarged fragmentary perspective view of a portion of the treating apparatus of the present invention and showing the front of the control housing thereof.

FIG. 3 is a fragmentary perspective view of the back of control housing of FIG. 2.

FIG. 4 is a fragmentary exploded view of the control housing of FIG. 2.

FIG. 5 is a somewhat enlarged, fragmentary vertical section of the conduit and nozzle assembly of the present invention.

FIG. 6 is a fragmentary side elevation of a portion of the treating apparatus of the present invention.

FIG. 7 is fragmentary side elevation of a portion of the treating apparatus of the present invention shown in a somewhat rotated attitude relative to that shown in FIG. 6.

FIG. 8 is a fragmentary vertical section of a second embodiment of the conduit and nozzle assembly of the treating apparatus of the present invention.

FIG. 9 is a fragmentary front elevation of the second embodiment of the treating apparatus of the present invention shown in a typical operative environment being worn by a person shown in phantom lines.

FIG. 10 is a fragmentary front elevation of the third embodiment of the treating apparatus of the present invention worn by a person shown in phantom lines.

### DETAILED DESCRIPTION OF THE INVENTION

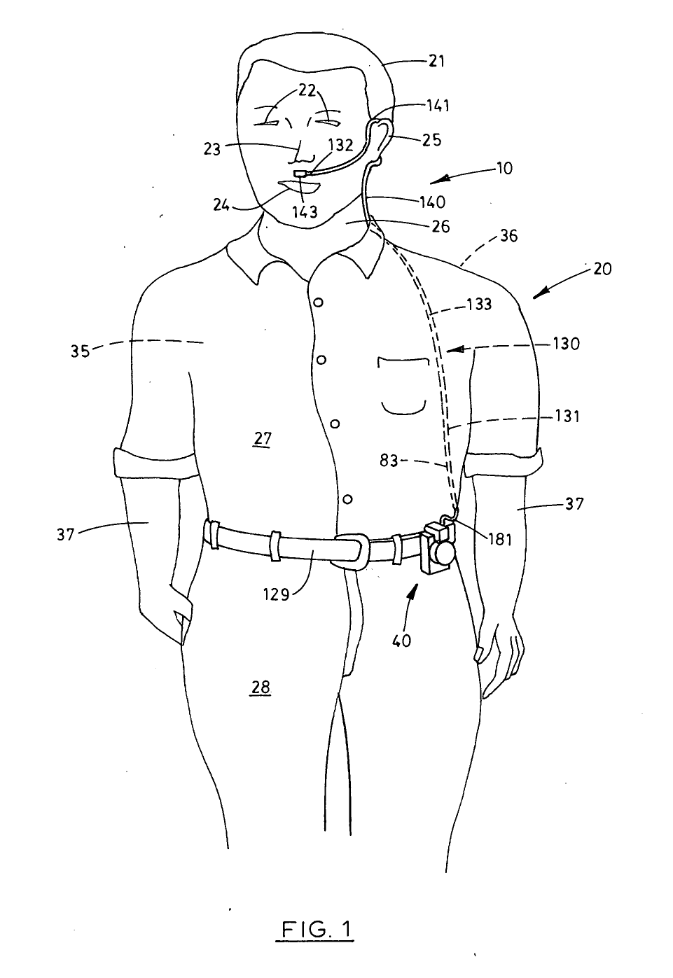

Referring more particularly to the drawings, the apparatus of the first embodiment of the present invention generally indicated by the numeral 10 in FIG. 1.

A plurality of embodiments of the treating apparatus 10 are shown and described herein. These embodiments are illustrative of a multiplicity of embodiments which are encompassed by the method and apparatus of the present invention. Since the embodiments shown and described herein have in some cases the same elements, for illustrative convenience the first embodiment of the apparatus of the present invention is first described herein. The first embodiment of the invention is similar to the subsequent embodiments in many respects; that is, those subsequent embodiments of the invention possess operational elements of various forms in addition to the general structure of the first embodiment of the invention.

### First Embodiment

The first embodiment of the treating apparatus of the present invention is shown in FIGS. 1, 2, 3, 4, 5, 6 and 7. Referring first to FIG. 1, the treating apparatus 10 is shown in a typical operative environment being worn by a person 20. For future reference and illustrative convenience, it will be understood that the person has bodily portions including a head 21, eyes 22, a nose 23 and a mouth 24. The person has ears 25 and a neck 26. Again, for illustrative convenience, insofar as clothing is concerned having any relationship to the invention hereof, the person is shown wearing a shirt 27, pants 28 and a belt 29.

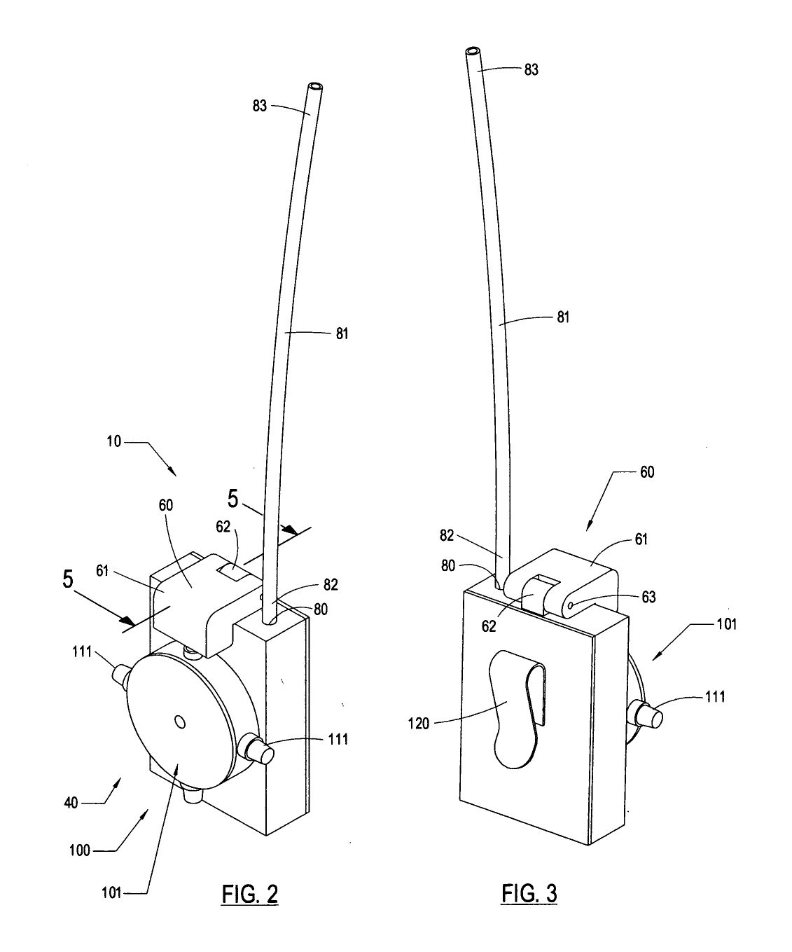

The treating apparatus 10 has a dispensing assembly or housing generally indicated by the numeral 40 in FIG. 1 and which is shown additionally in FIGS. 2, 3, 4 and 5. The dispensing assembly, as will be seen, can be manufactured in a variety of different forms. The dispensing assembly shown and described herein, however, is believed to have operative benefits, as will hereinafter be discussed in greater detail.

Referring more specifically to FIG. 4, the dispensing assembly 40 has a main housing 41 which, itself, has rectangular front wall 42 mounting four rectangularly related sidewalls 43. The front wall and four sidewalls define an interior chamber 44 therewithin. The main housing mounts a backwall 45 which thereby encapsulates the interior chamber. A pump assembly 50 is mounted within the interior chamber 44. The pump assembly can be of any suitable type having a power source, not shown. In the preferred embodiment, the pump is operable to pump an airborne substance, as will hereinafter be discussed in greater detail. The pump assembly has a pump housing 51 which mounts a discharge conduit 52 which extends upwardly from the pump housing. The discharge conduit terminates in an orifice 53 which also faces upwardly, as shown in FIG. 4. As noted, the pump assembly 50 is of any suitable type operable to release small quantities of a given airborne liquid when activated by a control system, not shown. The pump assembly can be operable to release such liquid, for example, in a continuous flow, or for given periods of time, or in pulses, or in any other manner desired.

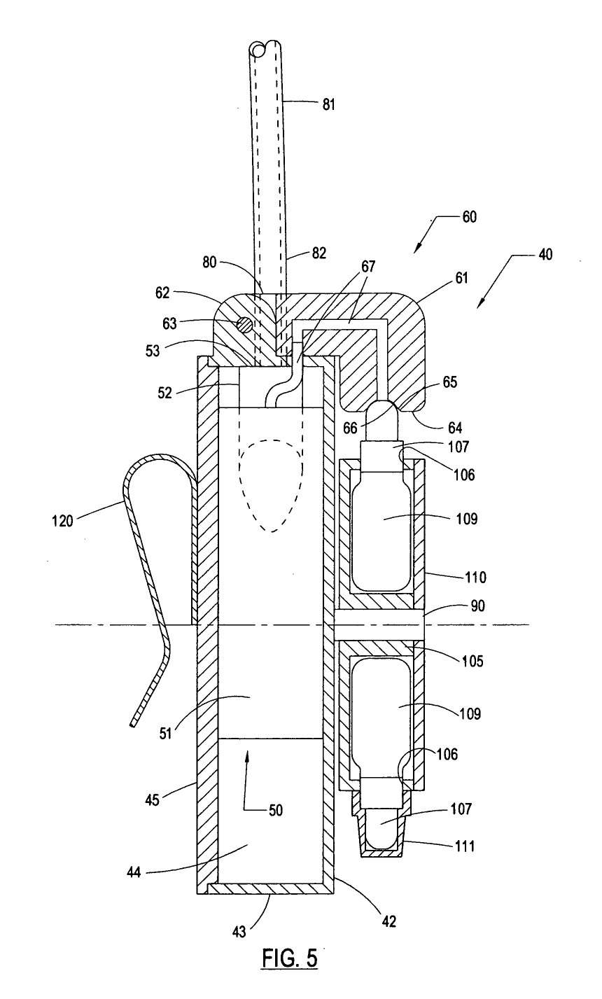

Referring more particularly to FIGS. 4 and 5, the dispensing assembly 40 has an intake mechanism 60 having an intake head 61. The intake head is mounted for pivotal movement on an intake head mount 62 about a pivot pin 63 extending therebetween. Thus, the intake head 61 is mounted for pivotal movement between a lowered position, shown in FIGS. 4 and 5, and a position pivotally elevated therefrom. The intake head has a lower surface 64 which faces downwardly in normal use, as shown in FIG. 5. The lower surface of the intake head has a transfer coupling 65 mounted thereon. The transfer coupling is generally concave in shape and has a seat 66 therein. A small substantially pin size conduit 67 extends through the intake head 61 from the seat, through the upper end wall 43 of the main housing 41 and into fluid supplying relation to the interior of the pump housing 51.

A discharge orifice 80 extends through the upper wall 43 of the main housing 41 in alignment with the orifice 53 of the discharge conduit 52 of the pump assembly 50. A flexible discharge conduit 81, having a proximal end portion 82 and a distal end portion 83, is mounted in and extends through the discharge orifice 80 and into the orifice 53 of the discharge conduit 52 in fluid transferring relation.

A mounting shaft 90 is mounted on and extends outwardly from the front wall 42 of the main housing 41 in substantially right angular relation thereto, as shown in FIG. 4. A fluid selection assembly 100 is mounted for rotational movement on the mounting shaft 90 of the main housing 41 for rotational movement about the mounting shaft. More specifically, the fluid selection assembly has a fluid carousel or wheel 101, shown in FIG. 4 in an exploded view, which is mounted for rotational movement on the mounting shaft. The wheel has a frame 102 which itself has a cylindrical ring 103. The cylindrical ring has cross members 104 which are mounted on the cylindrical ring and extend inwardly to a central hub 105. The central hub is mounted for rotational movement on the mounting shaft 90.

Four internally screwthreaded bores 106 extend through the cylindrical ring in spaced relation to each other, as shown in FIG. 4, about the cylindrical ring. Four nozzles 107 are individually mounted in the in the internally screwthreaded bores. Each nozzle communicates in operational relation to a quadrant chamber 108. A fluid receptacle 109 is mounted in each of the quadrant chambers in operable communication with its respective nozzle 107. An end plate 110 is mounted on and thereby forms an outer wall of the fluid wheel 101. Each of the nozzles is covered by a removable cap 111 so that each fluid receptacle is sealed until ready for use, as will hereinafter be discussed in greater detail.

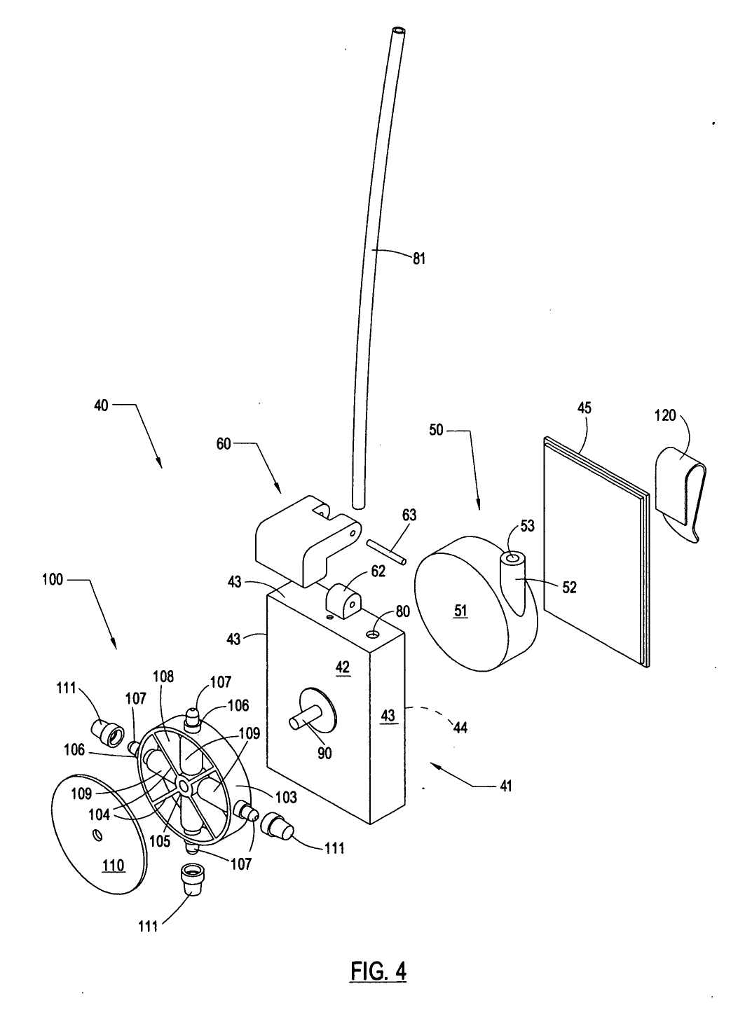

An attachment clip 120 is mounted on the backwall 45 of the main housing 41, as best shown in FIG. 3. The clip is employed removably to mount the main housing conveniently on the pants 28, or belt 29, or in any other convenient location for the person, as best shown in FIG. 1.

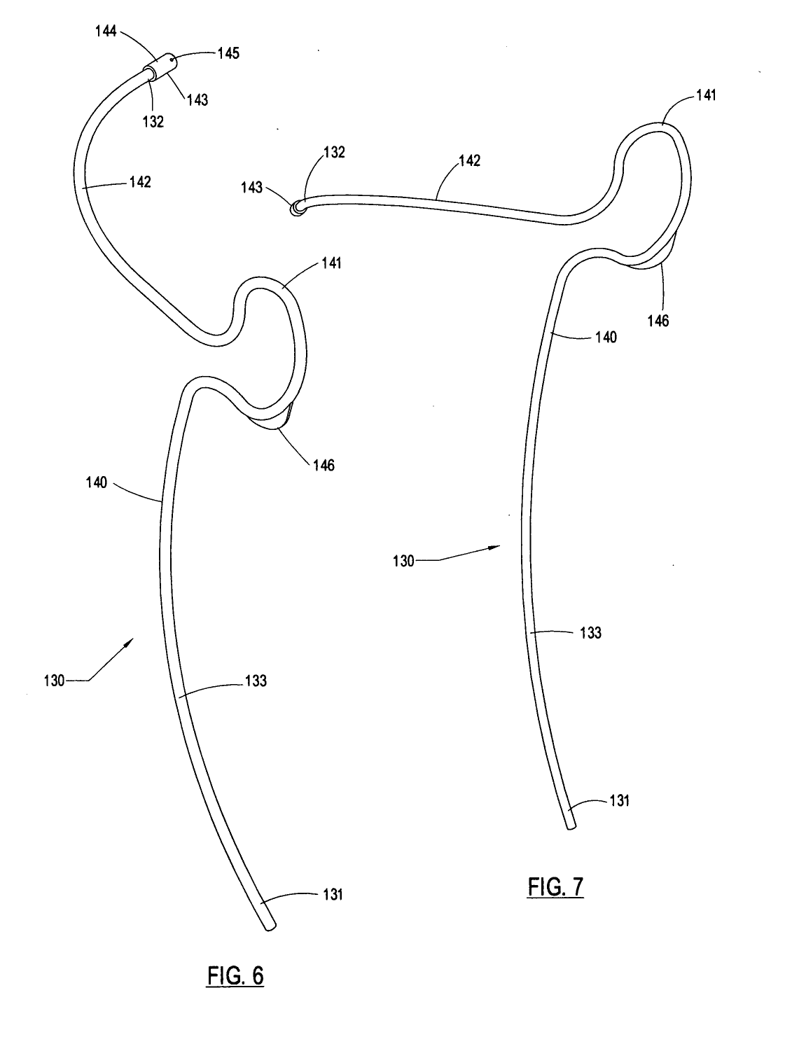

As also best shown in FIG. 1, the first embodiment of the subject invention has a main supply conduit 130. The main supply conduit has a first end portion 131 which is mounted in fluid receiving relation on the distal end portion 83 of the flexible discharge conduit. The main supply conduit has a second end portion 132 which is, perhaps, shown best in FIGS. 1 and 6. Intermediate the first end portion and the second end portion of the main supply conduit is a back segment 133. The main back segment preferably extends from the distal end portion 83 of the flexible discharge conduit 81 beneath the shirt 27 and up the back 36 of the person 20. The flexible discharge conduit 81 and main supply conduit 130 are thereby substantially concealed beneath the shirt of the person.

The main supply conduit 130 has a neck portion 140 which, in use, is adjacent to the neck of the person 20; an ear loop portion 141 which, in use, extends over the adjacent ear 25, as shown in FIG. 1; and a facial portion 142 which, in use, is extended along the face of the user and beneath the nose 23 of the person. A discharge nozzle assembly 143 is mounted on the terminal end of the facial portion beneath the nose. The discharge nozzle assembly has an adjustable dial 144 which can be turned, or rotated, to position a discharge aperture 145 thereof so as to face in a desired direction. As will subsequently be discussed in greater detail, the desired direction will usually be toward or away from the nose and mouth of the person depending upon the circumstances at hand. An ear loop flange 146 is mounted on the main supply conduit just beneath the ear loop portion, as best shown in FIGS. 6 and 7. The ear loop flange is shaped and positioned to engage the underside of the ear fitted therein to help support the ear loop in the correct location and attitude, as shown in FIG. 1.

### Second Embodiment

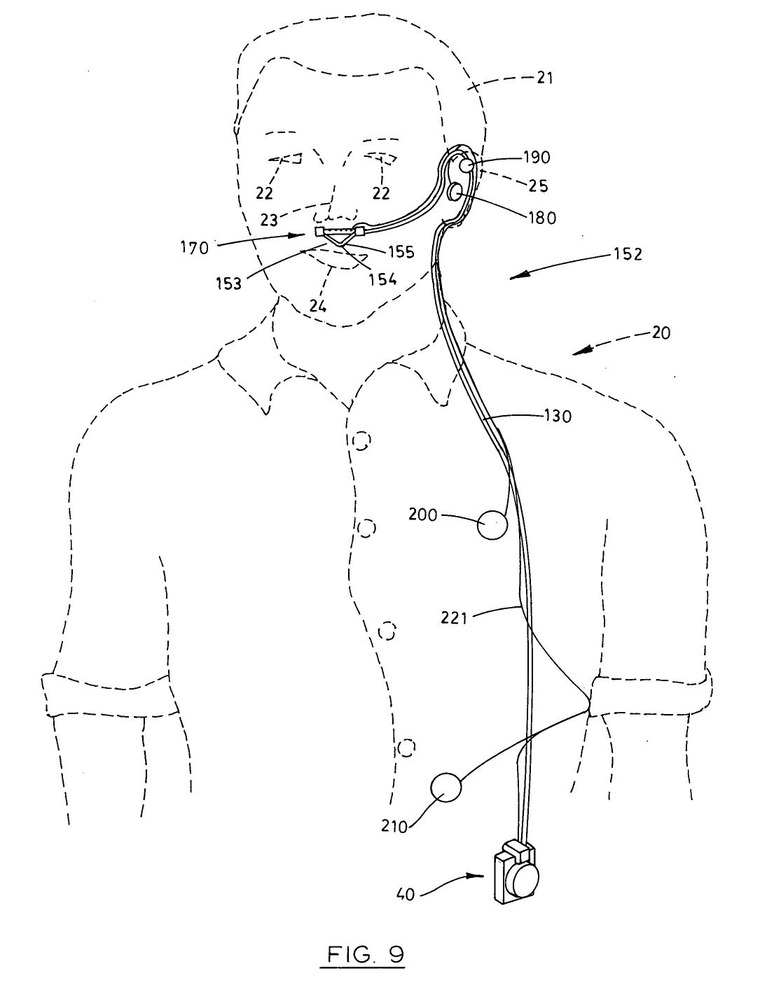

The second embodiment of the subject invention is shown in FIG. 9 and, as heretofore noted, is similar to the first embodiment of the subject invention in that it possesses all of the elements heretofore described with respect to the first embodiment with the exceptions hereinafter noted.

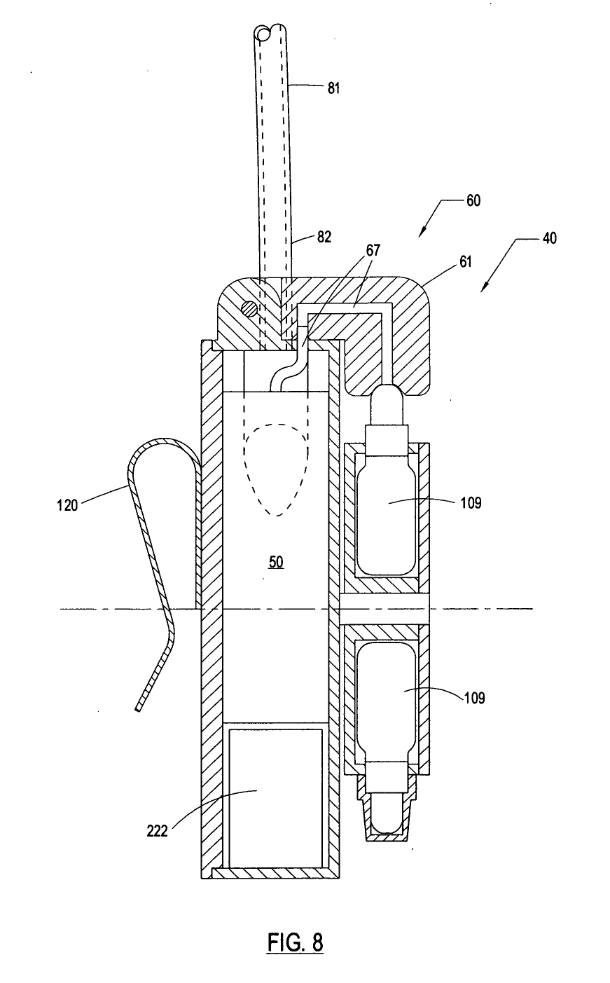

The discharge nozzle assembly 143 of the first embodiment is different from that of the second embodiment with respect to the specific form thereof. The discharge nozzle assembly 153 of the second embodiment has a V-shaped nose portion 154 shown in FIG. 8. As shown therein, the V-shaped nose portion has an aperture 155 at the apex of the V-shaped nose portion through which liquid medication of the like is discharged during operation.

For illustrative convenience, unless otherwise noted those portions of the second embodiment which are the same as in the first embodiment 10 are described herein only as believed necessary and helpful to a full understanding of the second embodiment. Except as otherwise noted it will be understood that the structure and operation are the same. Accordingly, the main supply conduit 130 is connected in fluid supplying relation to the V-shaped nose portion 154 of the discharge nozzle assembly 153. The V-shaped nose portion is pivoted on the main supply conduit to permit the aperture 155 thereof to be positioned, as in the first embodiment 10 so that the direction of fluid release can be projected toward the nose 23 of the person 20 or outwardly in the opposite direction away from the person's nose 23 and mouth 24. The main supply conduit in the second embodiment has a neck portion 140, and ear loop portion 141 and a facial portion 142 which are used as in the first embodiment 10 and heretofore described. The first end portion 131 or the main supply conduit 130 is connected to the distal end portion 83 of the flexible discharge conduit 81 in fluid receiving relation. Thus, the second embodiment appears in use roughly as in the case of the first embodiment as shown in FIG. 1. Except as otherwise will be noted, the dispensing assembly 40 of the second embodiment is the same as that of the first embodiment, such as shown in FIGS. 2, 3, 4 and 5.

The second embodiment of the present invention 152 has a laser light assembly generally indicated by the numeral 170 in FIG. 8. The laser light assembly is mounted on and extends between the arms of the V-shaped nose portion. Except as hereinafter discussed, the laser light assembly need not be described in greater detail because of its well-known structure and operation in the art.

With reference again to FIG. 9, the second embodiment of the present invention 152 has a thermometer or temperature gauge 180 which is received in the ear 25 of the person 20. The temperature gauge is held in place in the person's ear in the conventional fashion. The temperature gauge can be taped over to assist in holding it in place if desired.

The second embodiment of the present invention 152 has a pulse monitor 190 mounted on the back of the ear 25 of the person. The pulse monitor is operable to detect the pulse of the person and can adhesively be held in place.

The second embodiment 152 has a heart monitor 200 which is attached to the skin such as by a suitable adhesive, on the chest of the person 20 over his heart. The heart monitor is operable to detect the condition and operation of the heart of the person.

The second embodiment 152 has a back monitor 210 which is attached to the skin by a suitable adhesive. The back monitor is operable to detect the condition and operation of the back such as by registering, as it is designed to do, for example, muscle spasms, electrical impulses and the like.

The laser light assembly 170, temperature gauge 180, pulse monitor 190, heart monitor 200 and back monitor 210 are operably interconnected by an electrical system 220 which supplies electrical energy to the various monitors heretofore setforth and which records the information received from those monitors. For this purpose, the electrical system has an electrical conduit 221 operably interconnecting the monitors and a control system 222.

The control system 222 is preferably, although not necessarily, housed in the interior chamber 44 of the main housing 41 of the dispensing assembly 40 with the electrical conduit 221 connected thereto. The control system can either or both record the information received for subsequent downloading, or can contain a transmitter instantaneously to transmit the information to a receiver located in, for example, a doctor's office, hospital, or other medical care facility for use. The control system has a source electrical energy, not shown, for operation thereof.

### Third Embodiment

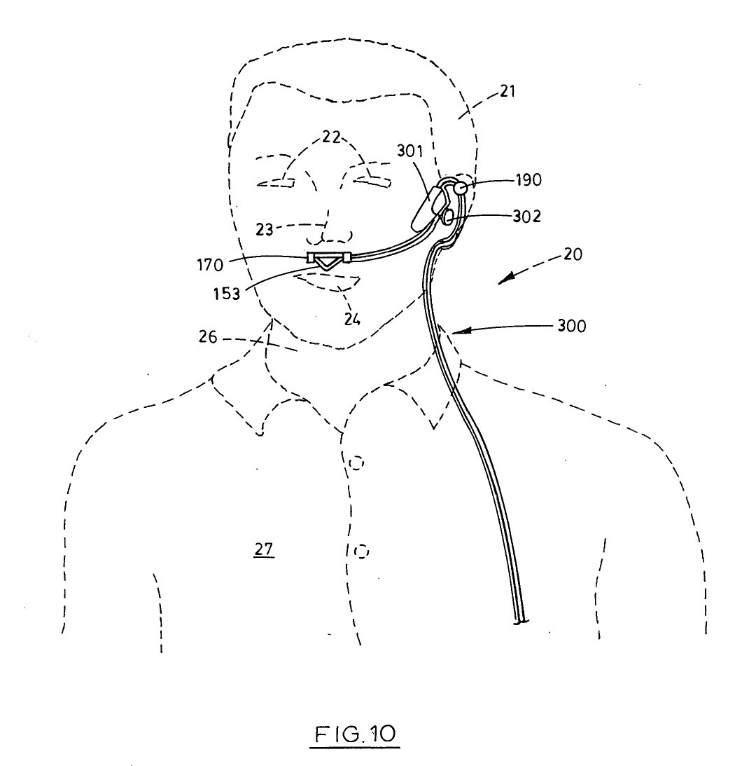

The third embodiment of the subject invention is generally indicated by the numeral 300 and is shown in FIG. 10. The third embodiment possesses the same elements as the first embodiment of the invention heretofore setforth. For illustrative convenience, it will be understood that the third embodiment also possesses the discharge nozzle assembly 153 and the pulse monitor 190 of the second embodiment of the invention 152.

Turning then, more specifically, to the differences between the third embodiment 10 and second embodiment 152, the primary distinction in the form shown in FIG. 9 is that it has a cellular telephone 301. The cellular telephone has an earpiece with in use us inserted into the ear 25 of the person 20 for use in a manner similar to a normal cellular telephone except as will subsequently be described herein in greater detail. The electrical conduit 221 of the electrical system 220 operatively interconnects the laser light assembly 170, the cellular telephone, the pulse monitor 190 and the control system 222 for operation as will hereinafter be described in greater detail.

### Operation

The operation of the described embodiments of the present invention are believed to be readily apparent and is briefly summarized at this point.

As in the case of the structure of the first, second and third embodiments of the present invention, the operation of these embodiments is the same for closely similar and various respects. Accordingly, a description of such operation is hereinafter described in summary form.

It will first be understood that the embodiments of the invention, or portions thereof can be used in various combinations as heretofore described and is believed to be readily apparent.

In the case of the first embodiment 10 of the subject invention, shown in FIGS. 1, 2, 3, 4, 5, 6, 7 and 8, the person 20 decides whether his personal health condition, or that of the other people with whom he may come into contact present a risk of exposure by either to a communicable unhealthful condition such, for example, as influenza. Where such a possibility exists, the person will choose to wear the treating apparatus of the first, second, or third embodiments as the situation warrants.

In the case of the first embodiment 10, the dispensing assembly 40 is attached, for example, to the belt 29, the interconnected flexible discharge conduit 81 and main supply conduit 130 are extended, preferably beneath the shirt 27 and upwardly against the neck 26. The ear loop portion 141 of the main supply conduit 130 is extended about, and thereby mounted on, the ear 25. The facial portion 142 is extended across the face of the person 20, as shown in FIG. 1 to position the discharge nozzle assembly 143 between the nose 23 and mouth 24, as shown in FIG. 1. If the person believes there is a risk of exposure to a communicable disease from others, the aperture 145 of the discharge assembly 143 is positioned so as to be directed outwardly, or away, from the person's face. If the person believes he may have communicable disease, he places the aperture to be directed toward his face including nose and mouth.

Subsequently, the person 20 operates the dispensing assembly 40 using the fluid wheel 101 to position the desired fluid receptacle 109 into snap-fitted engagement with the seat 66 of the intake head 61. As best shown in FIG. 5, the selected nozzle 107 is thereby disposed in fluid supplying relation to the pin size diameter conduit 67 for extraction of the fluid from the fluid receptacle by the pump assembly 50.

Each of the fluid receptacles 109 can contain any appropriate fluid such as, for example, air, a disinfectant, vitamins, antibacterial fluids, oxygen, insect repellants, water, or a medicine appropriate to treat the particular communicable disease, condition, or the like. Each fluid receptacle 109 can contain the same fluid or a fluid different form each of the other fluid receptacles. Thus, there is a significant flexibility of usage whereby the same or different fluids can be employed in any order and for any desired length of time.

Similarly, the pump assembly 50 can be programmed and/or operated to release a continuous spray, mist, or other dispersed configuration of the fluid from the aperture 145. Alternatively, the fluid can be released in pulses, or for limited periods of time, or any other mode of operation desired.

The dispersal of the mist away from the face of the user operates substantially to limit the user from being directly exposed to a communicable disease. The mist intercepts contaminated substances such as vapor, dust and other forms of contaminates so that they do not reach the user and communication is avoided.

All of the preferred embodiments of the subject invention possess the foregoing method and apparatus, although this is not required necessarily for all embodiments of the invention. However, both the second embodiment 152 and third embodiment 300 employ this method and apparatus as shown and described.

The second embodiment 152, best shown in FIGS. 8 and 9, employs additional systems as hereinafter setforth. The laser light assembly 170 emits light as the person breathes thereon. There is some recognition that laser light, which bounces back and forth multiple times, strikes the molecules contained in the breath. Spectrometry analysis show which frequencies of light were absorbed which, in turn, is an indirect measure of what molecules are in the sample. This information can be used then to detect evidence indicating the presence, for example, of cancer, asthma, diabetes, kidney malfunction and perhaps other diseases and conditions. It has been reported that experiments at the University of Colorado are being conducted on the use of laser light to test for such conditions. The laser light assembly 170 of the second embodiment of the present invention allows this analysis to be done during normal activities.

The second embodiment 152, as shown in FIG. 9 possesses additional health related benefits which may be characterized as including thermometer or temperature gauge 130 in the ear; the pulse monitor 190 behind the ear; and the heart monitor 210. Other types of monitors can be added, if desired, to those already included. The read out of the collected data is sent by way of the electrical system 220 and electrical conduit 221 to the control system.

As previously noted, this data can be stored in the control system for subsequent downloading. Alternatively and preferably, the data can immediately be transmitted by a suitable transmitter, not shown, to a designated monitoring location such as a doctor's office, hospital or other health care facility for prompt evaluation. The optimum and most practical mode of operation depends upon the physical condition of the person and the benefit of having immediate analysis of the data.

The third embodiment 300 of the subject invention is best shown in FIGS. 8 and 10. As previously discussed, the third embodiment of the subject invention may have all or any of the systems and elements of the first embodiment 152. Some portions thereof are shown in FIGS. 8 and 10. However, in the third embodiment the treating apparatus has a cellular telephone 301 placed in the ear and held in place using the earpiece 302. The presence of the cellular telephone in the third embodiment allows the person to telephone a suitable authority for assistance when an emergency occurs. Such authorities can, of course, be calling “911”, his doctor, a hospital or the like.

Therefore, the apparatus of the present invention is particularly well suited to use as a treating apparatus which provides protection against the spread of communicable diseases and the like; which is unobtrusive when worn by a person and yet which operates reliably and with an effectiveness not heretofore achieved in the art; which affords a flexibility of use allowing it to be tailored for the specific needs of the subject; which affords a broad range of benefits in use and yet which possesses a simplicity and adaptability in use; which is fully operational in a variety of embodiments; and which is otherwise entirely successful in achieving its operational objectives.

Although the invention has been herein shown and described in what is conceived to be the most practical and preferred embodiments, it is recognized that departures may be made therefrom within the scope of the invention which is not to be limited to the illustrative details disclosed. Having described my invention, what I claim as new and desire to secure by Letters Patent is:

1. A treating apparatus comprising a source of a flowable substance; means for releasing said flowable substance in a general direction relative to a target; and a propelling assembly operable to transport the flowable substance from said source to said releasing means whereby said flowable substance is propelled in said general direction relative to the target.

2. The treating apparatus of claim 1 wherein said releasing means is positionable for propelling the flowable substance in a selected general direction relative to the target.

3. The treating apparatus of claim 1 wherein said target is generally a face of the subject.

4. The treating apparatus of claim 1 wherein said target is generally facing away form the face of the subject.

5. The treating apparatus of claim 1 wherein said releasing of a flowable substance relative to a target is relative to the face of a person.

6. The treating apparatus of claim 5 wherein said flowable substance is a liquid and the releasing means releases said flowable substance substantially as a mist.

7. The treating apparatus of claim 6 wherein said flowable substance is a liquid treating agent operable to reduce susceptibility of said person to a condition in the vicinity of the person.

8. The treating apparatus of claim 6 wherein said flowable substance is a liquid treating agent operable to reduce the susceptibility of other people in the vicinity to a condition of said person.

9. The treating apparatus of claim 6 wherein said releasing means includes a pressurized source of said flowable substance, a conduit operably extending form the pressurized source to an area in proximity to the face of said person and a mist nozzle mounted on the terminal end of the conduit operable to release the liquid as a mist and positionable relative to said terminal end for selectable positioning thereof to control said direction of release.

10. The treating apparatus of claim 9 wherein said pressurized source has a housing containing a pressure chamber, a battery for powering said pressurized source and a container containing said treating agent and disposed to release the treating agent into the pressure chamber for delivering to said mist nozzle for release thereby.

11. The treating apparatus of claim 10 wherein said container housing the treating agent is mounted on a movable dispensing member containing a plurality of said containers housing different treating agents and said dispensing member is movable by said person to select the desired treating agent by positioning said container housing the desired treating agent in dispensing relation to the pressure chamber.

12. A treating apparatus comprising a source of means for treating a physical condition; means for directing said means relative to a target; and a propelling assembly operable to release said treating means along a predefined path relative to said target to treat said physical condition.

13. The treating apparatus of claim 12 wherein said means for treating a physical condition is a laser beam generated by said source.

14. A treating apparatus comprising a source of means for treating a physical condition of a person; means for directing said means relative to the nose of the person; a propelling assembly operable to release said treating means in the general direction of the nose; a probe in the ear of the person continuously to read the temperature of said person; means for reading the pulse rate and heart rate of the person; and communicating means for transmitting said information to a location to monitor the information.
